(19) United States
(12) Patent Application Publication
(10) Pub. No.: US 2003/0211150 A1
(43) Pub. Date: Nov. 13, 2003

(54) Immediate release tablet containing naproxen sodium

(75) Inventors: Ahmad Al-Ghazawi, Amman (JO); Kalyan S. Nuguru, Deptford, NJ (US); Dan F. Giambattisto, Brewster, NY (US)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 10/300,938

(22) Filed: Nov. 21, 2002

Related U.S. Application Data
(60) Provisional application No. 60/421,153, filed on Nov. 26, 2001.

(51) Int. Cl. A61K 9/22; A61K 31/192

(52) U.S. Cl. 424/468; 514/569

(57) ABSTRACT

The present invention relates to a new tablet having an improved dissolution rate of Naproxen Sodium comprising Naproxen Sodium and spray-dried mannitol. The invention further relates to a method of manufacturing said tablet.

The present invention is directed to a new tablet having improved dissolution rate of Naproxen Sodium and to a method of manufacturing of said formulation.

Naproxen Sodium is a well-known anti-inflammatory, analgesic, and antipyretic agent used in the symptomatic treatment of acute and chronic rheumatoid arthritis, osteoarthritis, primary dysmenorrhea and for relief of mild to moderate pain. It has been approved in many countries around the world for almost two decades and has a very safe risk-benefit profile. In the United States it is sold, for example, under the trade name ALEVE® (distributed by Bayer Corporation, Consumer Care Division, Morristown, N.J.) and as generic Naproxen Sodium tablets (distributed by Rite-Aid Corporation, Harrisburg, Pa.). The chemical name of Naproxen Sodium is (−)-6-methoxy-alpha-methyl-2-napthaleneacetic acid, sodium salt.

Especially in case of using of naproxen tablets for the treatment of pain a fast onset of action is required and highly desired.

WO 98/35666 A1 describes tablets comprising naproxen nanoparticles having adsorbed on its surface a surface modifying agent and further excipients. Naproxen nanoparticles are obtained by wet grinding of naproxen together with the surface modifying agent, separating the surface modified particles obtained, spray drying and sieving. Accordingly, production of solid formulation is complicated and costly.

WO 00/13672 A1 assigned to the same company as WO 98/35666 A1 describes a solid nanoparticulate formulation obtained by compression of naproxene nanoparticles having adsorbed on its surface a surface modifying agent together an alkali agent. As WO 00/13672 A1 does not contain any details about production of nanoparticles, it is apparent that they are produced by the same procedure as described in WO 98/35666 A1. Accordingly, the same situation as described for WO 98/35666 A1 apply for WO 00/13672 A1.

EP 0 636 363 B1 disclose a rapidly disintegrating tablet dissolving in the mouth at least containing an active ingredient coated with a taste masking coating, a carbohydrate and a binder. In general, such formulations are sensitive against breakage and require a very careful handling, including manufacturing, packaging, transport and administration. Moreover, naproxen is mentioned as active ingredient, but no embodiment is presented.

It is the objective of the present invention to provide a new formulation for Naproxen Sodium having a fast dissolution rate in vitro as well as a fast absorption rate and a decreased fast/fed variability in vivo, which can be produced easily and cheaply.

It has been found that a solid Naproxen Sodium formulation having a fast dissolution could be provided when a mixture comprising of Naproxen Sodium and spray-dried mannitol is compressed into tablets. Accordingly, the present invention is directed to a tablet for immediate release of Naproxen Sodium comprising Naproxen Sodium and spray-dried mannitol.

Preferably the spray-dried mannitol used as ingredient for preparation of the tablet has a mass median particle diameter from 75 to 300 microns, a flowability from 25 to 45 degrees, a loose density from 0.35 to 0.75 g/ml and a tapped density from 0.45 to 0.85 g/ml.

More preferably the spray-dried mannitol used as ingredient for preparation of the tablet has a mass median particle diameter from 75 to 300 microns, a flowability from 25 to 40 degrees, a loose density from 0.40 to 0.60 g/ml and a tapped density from 0.50 to 0.75 g/ml.

Most preferably the spray-dried mannitol used for preparation of the tablet has a mass median particle diameter from 75 to 250 microns, a flowability of about 31 degree, a loose density of about 0.51 g/ml and a tapped density of about 0.60 g/ml. Spray-dried mannitol having said physical properties is marketed by Merck KGaA under the trade name Parteck M200.

“Flowability” as used herein is described by the angle of repose which is measured from a heap carefully built up by dropping the material through a vibrating screen and glass funnel onto a horizontal plate by using a mechanical lever. All values given in the present application for “flowablilty” are to be understood as measured by the procedure defined in ISO 4324.

“Loose density” as used herein is defined as apparent density of the powder and is calculated as ratio of mass (weight) to volume wherein mass is defined by the specific amount of powder placed in a calibrated cylinder and volume is defined as the volume of this specific calibrated cylinder. All values given in the present application for “loose density” are to be understood as measured by the procedure defined in DIN EN ISO 60.

“Tapped density” as used herein is the ratio of mass (weight) to volume obtained by the same procedures as described for “loose density” with the only difference that the volume used for calculation of tapped density is determined after tapped the cylinder for 1250 times. All values given in the present application for “tapped density” are to be understood as measured by the procedure defined in DIN EN ISO 787-11.

The spray-dried mannitol is present in the tablet of the present invention in an amount from 10 to 90% by weight, preferably in an amount from 30 to 70% by weight.

“Spray-dried mannitol” as used herein is obtained by spray-drying of solutions and/or suspensions of mannitol. To improve process of spray-drying and/or physico-chemical characteristics of the spray-dried mannitol the solutions/suspensions used for its producing can also contain a further polyol like sorbitol or lactitol in an amount of 0.1 to 20% by weight (relating to the total amount of polyol). Accordingly, spray-dried mannitol used for preparation of the tablet of the present invention may also contain up to 20% by weight of a further polyol. Preferably the spray-dried mannitol used contain 0.5 to 2.0% by weight of the polyol, more preferably about 1% by weight of the polyol. Preferably the polyol which can be further present in the spray-dried mannitol is sorbitol.

A process usable for production of spray-dried mannitol as used herein is described in WO 97/39739 A2.

Advantageously, the tablet of the present invention further contain a lubricant and/or glidant. Lubricants and/or glidants are intended to improve processing of mixture of Naproxen Sodium used for the manufacture of the tablet and, therefore, to achieve a better quality of tablets. Usable lubricants/glidants include, for example, fumed or colloidal silica, stearates (e.g. magnesium stearate, calcium stearate, stearic acid), talc, polyethylene glycol, starch, sodium stearyl fumarate, magnesium lauryl sulphate, sodium lauryl sulphate. Magnesium stearate or sodium stearyl fumarate are preferred. If used, lubricants/glidants are present in a ratio from 0.1 to 10% by weight.

Moreover, further auxiliary substances such as diluents or disintegrants may be present in the tablet of the present invention.

Usable diluents are, for example, dextrose, dicalcium phosphate, lactose, microcrystalline cellulose, sodium chloride or sucrose.

Usable disintegrants are, for example, starch, cation exchange resin, polyvinylpyrrolidone, modified starch, microcrystalline material (e.g. microcrystalline cellulose), alginic acid, sodium starch glycolate, modified cellulose or gum. Diluents and/or disintegrants may be present in the tablet of invention in an amount from 10 to 90% by weight and/or from 5 to 90% by weight, respectively.

The tablet of the present invention can be easily manufactured by mixing its ingredients and compressing the mixture to tablets. Accordingly, the present invention is also directed to a process for the manufacture of the tablet of the present invention which is characterized in that the ingredients of present in said tablet are mixed together and compressed to tablets.

Compression of the mixture containing all ingredients of the tablet of the present invention to tablets can be performed by using conventional tabletting machines. Although parameters of tabletting procedure like compression force also effects dissolution rates of the tablets, dissolution properties of the tablets are mainly affected by the composition of the mixture used for compression. Accordingly, the present invention is also directed to the mixture usable for the manufacture of the tablet of the present invention, characterized in that the mixture comprise the ingredients which are present in the tablet.

The following examples are given to illustrate the present invention. It should be understood, however, that the invention is not to be limited to the specific conditions or details described in these examples.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

The entire disclosure of all applications, patents and publications, cited above and below and provisional application No. ______, filed Nov. 26, 2001 is hereby incorporated by reference.

### EXAMPLE 1

### Formulation A

Composition of immediate release tablets.

| | | |
|---|---|---|
| Naproxen Sodium | 220 | mg |
| Median Particle Size 91 microns | | |
| Parteck M200 (spray-dried Mannitol) | 469.5 | mg |
| PRUV ™ (Sodium Stearyl Fumarate) | 10.5 | mg |

Blending:

Ingredients were passed through a #20 mesh to de-lump. Naproxen Sodium and 50% of Parteck M200 were added to a 2-Qt V-blender and blended for 4 minutes.

Rest of the Parteck M200 was added and blended for an additional 4 minutes.

Sodium Stearyl Fumarate (previously sieved through a #40 mesh or finer screen) was added to the blender and blended for 4 more minutes.

Blend was discharged and stored until tabletted.

Tabletting:

Tablets were prepared on a Kilian 28A tablet press. Tablet press speed was set at 30 rpm.

Punches used were 9/16 inches, S.S. concave, bevel edged.

Compression force was adjusted to attain a tablet hardness of around 8 KP.

Tablets were collected and stored for testing.

### EXAMPLE 2

### Formulation B

| | |
|---|---|
| Naproxen Sodium | 220 mg |
| (Median Particle Size 60 microns) | |
| Parteck M200 (spray-dried Mannitol) | 671 mg |
| Mg-Stearate | 9 mg |

Ingredients were mixed and compressed to tablets using the same procedure as described in Example 1.

### EXAMPLE 3

### Formulation C

| | | |
|---|---|---|
| Naproxen Sodium | 220 | mg |
| Median Particle Size 91 microns | | |
| Parteck M200 (spray-dried Mannitol) | 469.5 | mg |
| Mg-Stearate | 7 | mg |
| Cab-O-Sil | 3.5 | mg |

Ingredients were mixed and compressed to tablets using the same procedure as described in Example 1.

### COMPARATIVE EXAMPLE

### Formulation D

With the exemption that granular mannitol was used instead of spray-dried mannitol, the same ingredients as used for Formulation B (Example 2) were mixed and compressed to tablets using the same procedure as described in Example 2.

### EXAMPLE 4

### In Vitro Release of Naproxen Sodium

Dissolution test was performed according USP-24 apparatus-2 (Paddles) using 900 ml phosphate buffer pH 7.4 at 37° C., speed of rotation was 50 rpm.

Phosphate buffer pH 7.4 (0.1 M) was prepared by dissolving 2.62 grams of monobasic sodium phosphate and 11.50 grams of anhydrous dibasic sodium phosphate in water to make 1000 ml.

Release rate was determined photometrically at λ=332 nm.

Dissolution rates were determined for formulations A, B, C described in Examples 1 to 3, and, for comparison purposes, for formulation D produced by using granular mannitol and for two commercially available immediate release formulations Brand (Aleve®) from Bayer and Generic from Rite-Aid. Dissolution data obtained are presented as percent dissolved in Table 1 (Mean±standard deviation of at least 3 samples/readings; n.d.=not determined).

According to the product description, Aleve® contains 220 mg of Naproxen Sodium, microcrystalline Cellulose (MCC), Povidone, Magnesium stearate, Talc and Opadry-YS-1-4215.

Rite-Aid contains 220 mg of Naproxen Sodium, MCC, Croscarmellose sodium, Povidone, Talc, Colloidal silicon dioxide, Magnesium stearate and Opadry-YS. (which contains HPMC, PEG, Polysorbate-80, Titanium dioxide, F, D & C Blue #2 Al. lake)

TABLE1
% Dissolved (± S.D.)

| | | | | | | Generic |
|---|---|---|---|---|---|---|
| Time | Formul. | Formul. | Formul. | Formul. | | from |
| (min) | A | B | C | D | Aleve ® | Rite-Aid |
| 5.0 | 92.4 | n.d. | 51.1 | n.d. | 29.3 | 24.1 |
| | (1.0) | | (3.4) | | (3.0) | (1.3) |
| 10 | 92.9 | 96.0 | 78.4 | 60.5 | 65.8 | 52.5 |
| | (1.3) | (0.9) | (4.6) | (6.6) | (3.8) | (1.7) |
| 20 | 92.0 | n.d. | 96.5 | n.d. | 95.7 | 87.5 |
| | (1.0) | | (0.7) | | (1.0) | (0.2) |
| 30 | 91.1 | 95.0 | 95.7 | 90.7 | 95.2 | 90.4 |
| | (0.9) | (0.8) | (0.8) | (3.0) | (0.4) | (5.1) |
| 60 | 90.3 | 94.2 | 94.7 | 89.6 | 93.7 | 92.3 |
| | (0.9) | (0.8) | (0.7) | (2.8) | (0.2) | (1.2) |

The results clearly shows that the tablets of the present invention have an improved dissolution rate compared to the tablets already on the market. Moreover, the tablet of the present invention containing spray-dried mannitol has also a faster dissolution rate compared to the tablet containing granular mannitol (instead of spray-dried mannitol).

Bioavialiability Study

Comparative, randomised, single dose, two-way crossover bioavailability study of Naproxen Sodium 220-mg oral tablets was done in 24 healthy Rabbits under fasting and fed conditions using formulation A according to Example 1 and Aleve®. Study Drugs (Naproxen Sodium 220-mg tablets) were administered in an oral dose of quarter a-tablet (55 mg) according to a randomisation plan. For all subjects the washout period was 7 days. Twelve (1-12) healthy Rabbits were used in the fasted state study, while another twelve (13-24) healthy Rabbits were used in the fed state study. Blood samples were taken at timed intervals up to 4 hours after dosing and assayed for Naproxen by high performance liquid chromatography—(HPLC). Pharmacokinetic parameters were determinted by standard non-compartmental analysis and then subjected to statistical analysis of variance test using Kinetica program.

Pharmakokinetic data obtained are summarized in Table 2.

TABLE 2

| | |
|---|---|
| fasted state | fed state |

| Parameter | Formul. A | Comp | Formul. A | Comp |
|---|---|---|---|---|
| AUC₍₀₋₄ₕ₎ | 88.4 | 40.6 | 129.8 | 32.1 |
| [μg/ml h] ± | 42.3 | 32.7 | 71.2 | 30.8 |
| S.D. [%] | | | | |
| Cₘₐₓ | 32.9 | 16.7 | 56.2 | 15.2 |
| [μg/ml] ± | 17.5 | 12.7 | 28.1 | 12.2 |
| S.D. [%] | | | | |
| tₘₐₓ | 1.08 | 1.50 | 0.80 | 0.92 |
| [hours] ± | 0.88 | 1.58 | 1.27 | 1.20 |
| S.D. [%] | | | | |

The data clearly show that time to peak plasma concentration (tₘₐₓ) was shorter and that maximum plasma concentration (cₘₐₓ) was increased for the tablet of the present invention compared to Aleve® of Naproxen Sodium, both in fed and in fasted state. Further, the variability of time to peak plasma concentration in fasted state compared to fed state was reduced. Accordingly, the tablet of the present invention leads to a faster onset of action and to a stronger effect, which is especially advantageously when the the tablet is used for the treatment of pain. Moreover, as bioavailiability (AUC) of the tablet of the present invention is increased the same effect can be obtained with a lower dose of Naproxen Sodium so that the amount of Naproxen Sodium present in the tablet can be reduced.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Tablet for immediate release of Naproxen Sodium comprising Naproxen Sodium and spray-dried mannitol

2. Tablet according to claim 1, characterized in that the spray-dried mannitol used for preparation of the tablet has a mass median particle diameter from 75 to 300 microns, a flowability from 25 to 45 degrees, a loose density from 0.35 to 0.75 g/ml and a tapped density from 0.45 to 0.85 g/ml

3. Tablet according to claim 1 and/or 2 characterized in that the spray-dried mannitol used for preparation of the tablet has a mass median particle diameter from 75 to 300 microns, a flowability from 25 to 45 degrees, a loose density from 0.40 to 0.60 g/ml and a tapped density from 0.50 to 0.75 g/ml

4. Tablet according to one or more of claims 1 to 3 characterized in that the spray-dried mannitol used for preparation of the tablet has a mass median particle diameter from 75 to 250 microns, a flowability of about 31 degree, a loose density of about 0.51 g/ml and a tapped density of about 0.60 g/ml

5. Tablet according to one or more of claims 1 to 4 characterized in that the tablet further contain a lubricant and/or glidant

6. Tablet according to claim 5 characterized in that the lubricant is magnesium stearate and/or sodium stearyl fumarate

7. Tablet according to one or more of claims 1 to 6 characterized in that the spray-dried mannitol is present in the tablet in an amount from 10 to 90% by weight

8. Tablet according to claim 7 characterized in that the spray-dried mannitol is present in the tablet in an amount from 30 to 70% by weight

9. Mixture usable for the manufacture of a tablet for immediate release of Naproxen Sodium characterized in that the mixture comprise the ingredients as specified in one or more of claims 1 to 8

10. Process for the manufacture of the tablet according to one or more of claims 1 to 8 characterized in that the ingredients present in said tablet are mixed together and compressed to tablets
